(19) United States
(12) Patent Application Publication
(10) Pub. No.: US 2015/0290130 A1
(43) Pub. Date: Oct. 15, 2015

(54) PREPARATION METHOD OF POLYMERIC MICELLES COMPOSITION CONTAINING A POORLY WATER-SOLUBLE DRUG

(71) Applicant: SAMYANG BIOPHARMACEUTICALS CORPORATION, Seoul (KR)

(72) Inventors: Min Hyo SEO, Seo-gu (KR); Sa Won LEE, Yuseong-gu (KR)

(21) Appl. No.: 14/750,371

(22) Filed: Jun. 25, 2015

Related U.S. Application Data
(63) Continuation-in-part of application No. 13/140,363, filed on Jun. 16, 2011.

(30) Foreign Application Priority Data
Dec. 26, 2008 (KR) 10-2008-0134506

(51) Int. Cl. A61K 9/107; A61K 9/51; A61K 9/19; A61K 31/337

(52) U.S. Cl. CPC A61K 9/1075; A61K 31/337; A61K 9/5153; A61K 9/19

(57) ABSTRACT

Provided is a method for preparing a drug-containing polymeric micelle composition, which includes: dissolving a drug and an amphiphilic block copolymer into an organic solvent; and adding an aqueous solution to the resultant mixture in the organic solvent to form polymeric micelles, wherein the method requires no separate operation to remove the organic solvent prior to the formation of micelles. The method for preparing a drug-containing polymeric micelle composition is simple, reduces the processing time, and is amenable to mass production.

### CROSS-REFERENCE TO RELATED APPLICATION

This application is a continuation-in-part of U.S. patent application Ser. No. 13/140,363 filed Jun. 16, 2011, which is a National Stage of International Application No. PCT/KR2009/003521 filed Jun. 29, 2009, which claims priority to Korean Patent Application No. 10-2008-0134506 filed Dec. 26, 2008. The entire disclosures of all of the above applications are incorporated herein by reference.

### TECHNICAL FIELD

This disclosure relates to a method for preparing a drug-containing polymeric micelle composition.

### BACKGROUND ART

Submicronic particulate drug delivery systems using biodegradable polymers have been studied for the purpose of intravenous administration of drugs. Recently, it has been reported that nanoparticle systems and polymeric micelle systems using biodegradable polymers are useful technological systems that modify the in vivo distribution of a drug administrated through a vein to reduce undesired side effects and to provide improved efficiency. Additionally, because such systems enable targeted drug delivery, they achieve controlled drug release to a target organ, tissue or cell. In fact, such systems are known to have excellent compatibility with body fluids and to improve the solubilization ability of a poorly water-soluble drug and the bioavailability of a drug.

Recently, there has been reported a method for preparing block copolymer micelles by bonding a drug chemically to a block copolymer containing a hydrophilic segment and a hydrophobic segment. The block copolymer is an A-B type diblock copolymer polymerized from a hydrophilic segment (A) and a hydrophobic segment (B). Such drugs as Adriamycin or Indomethacin may be physically encapsulated within the cores of the polymeric micelles formed from the block copolymer, so that the block copolymer micelles may be used as drug delivery systems. However, the polymeric micelles formed from the block copolymer cause many problems in the case of in vivo applications, since they cannot be hydrolyzed but are decomposed merely by enzymes in vivo, and they have poor biocompatibility by causing immune responses, or the like.

Therefore, many attempts have been made to develop core-shell type drug delivery systems having improved biodegradability and biocompatibility.

For example, diblock or multiblock copolymers including polyalkylene glycol as a hydrophilic polymer and polylactic acid as a hydrophobic polymer are known to those skilled in the art. More particularly, acrylic acid derivatives are bonded to the end groups of such diblock or multiblock copolymers to form copolymers. The resultant copolymers are subjected to crosslinking to stabilize the polymeric micelles.

However, methods for preparing such diblock or multiblock copolymers have difficulties in introducing crosslinkers to the hydrophobic segments of A-B or A-B-A type diblock or triblock copolymers so that the polymers are in stable structures via crosslinking. Additionally, the crosslinkers used in the above methods cannot ensure safety in the human body because the crosslinkers have no application examples in the human body. Furthermore, the crosslinked polymers cannot be decomposed in vivo, and thus cannot be applied to in vivo use.

In addition to the above, known methods for preparing a polymeric micelle composition include an emulsification process, a dialysis process and a solvent evaporation process. The emulsification process includes dissolving polylactic acid into a water immiscible solvent, adding a drug to the polymer solution so that the drug is completely dissolved therein, and further adding a surfactant thereto to form an oil-in-water emulsion, and evaporating the emulsion gradually under vacuum. Since the emulsification process requires equipments for forming the emulsion, it is difficult and sophisticated to set the processing conditions. Additionally, since the emulsification process includes evaporation of an organic solvent, it requires a long period of processing time. Meanwhile, the dialysis process requires consumption of a large amount of water and needs a long period of processing time. Further, the solvent evaporation process requires a equipment, such as a rotary reduced-pressure distillator, for removing a solvent, and it takes a long period of time to remove the solvent completely. Moreover, the solvent evaporation process essentially includes an operation of exposing reagents to a high temperature for a long period of time, and thus it may cause such problems as decomposition of pharmaceutically active ingredients or decrease of pharmacological effects.

### DETAILED DESCRIPTION

### Technical Problem

Provided is a method for preparing a drug-containing polymeric micelle composition.

### Technical Solution

Disclosed herein is a method for preparing a drug-containing polymeric micelle composition, which includes: dissolving a drug and an amphiphilic block copolymer into an organic solvent; and adding an aqueous solution to the resultant mixture in the organic solvent to form polymeric micelles, wherein the method requires no separate operation to remove the organic solvent prior to the formation of micelles.

### Advantageous Effects

The method for preparing a drug-containing polymeric micelle composition disclosed herein is simple, reduces the processing time, and is amenable to mass production. In addition, the method allows preparation of a drug-containing polymeric micelle composition at low temperature or room temperature, thereby improving the stability of a drug.

### MODE FOR INVENTION

Exemplary embodiments now will be described more fully hereinafter with reference to the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for the purpose of describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In one aspect, there is provided a method for preparing a drug-containing polymeric micelle composition, which includes:

dissolving a poorly water-soluble drug and an amphiphilic block copolymer into an organic solvent; and

adding an aqueous solution to the resultant mixture in the organic solvent to form polymeric micelles,

wherein the method requires no separate operation to remove the organic solvent prior to the formation of micelles.

More particularly, according to the method for preparing a drug-containing polymeric micelle composition disclosed herein, a drug and a polymer are dissolved in an organic solvent, particularly a water miscible organic solvent, and then an aqueous solution is added thereto to form polymeric micelles in the mixed organic solvent/water. Therefore, the polymeric micelle composition obtained from the method disclosed herein includes a drug and an amphiphilic block copolymer. In addition, the method requires no separate operation to remove the organic solvent used for preparing polymeric micelles prior to the formation of micelles.

The presence of an organic solvent in a micelle solution during the formation of micelles facilitates de-association of micelles due to a high affinity of the hydrophobic portion of the amphiphilic polymer micelles to the organic solvent, thereby accelerating precipitation of hydrophobic drug molecules. For this reason, processes for preparing polymeric micelles known to date include dissolving a drug and an amphiphilic polymer into an organic solvent, removing the organic solvent, and adding an aqueous solution thereto to form micelles. However, such processes need a long period of processing time to remove the organic solvent, and require an additional equipment, such as a distillator under reduced pressure. In addition, the organic solvent may still remain partially in the reaction system even after removing it. Further, the drug may be decomposed as it is exposed to high temperature for a long time during the removal of the organic solvent.

According to one embodiment of the method disclosed herein, micelles may be formed at low temperature instead of removing the organic solvent at high temperature during the formation of micelles. In general, when polymeric micelles are heated, associated amphiphilic polymers become susceptible to de-association as the unimer of the amphiphilic polymer get an increased kinetic energy. As a result, hydrophobic drug molecules present in the hydrophobic core of micelles are in contact easily with the aqueous phase, thereby causing formation and precipitation of drug crystals. On the contrary, the method disclosed herein requires no separate solvent evaporation before forming micelles, thereby simplifying the overall process and preventing the decomposition of a drug. Further, the method disclosed herein is carried out at low temperature so that the resultant polymeric micelles maintain their stability.

Even though the organic solvent is not removed but exists at a certain concentration or higher as in the method disclosed herein, forming micelles while maintaining low temperature may prevent precipitation of a drug. This is because the amphiphilic polymer and organic solvent molecules have a decreased dynamic energy under such a low temperature, and thus the drug present in the hydrophobic segment of the amphiphilic polymeric micelles may not be easily exposed to the aqueous phase.

In one embodiment, the polymer micelles are formed by adding an aqueous solution to the drug/amphiphilic polymer mixture in an organic solvent at a temperature of 0° C. or more and less than 40° C., and particularly 5-20° C.

In another embodiment, although there is no particular limitation in the particular type of the drug encapsulated within the micelle structures of the amphiphilic block copolymer, the drug may be a poorly water-soluble drug. For example, the drug may be a poorly water-soluble drug having a solubility of 100 mg/mL or less to water. This is because the method disclosed herein is designed to provide a composition for administering a poorly water-soluble drug to the human body by encapsulating the drug within micelle structures.

In still another embodiment, the poorly water-soluble drug may be selected from anticancer agents. Particularly, the poorly water-soluble drug may be selected from taxane anticancer agents. Particular examples of the taxane anticancer agents may include paclitaxel, docetaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel or a mixture thereof. More particularly, the taxane anticancer agent may be paclitaxel or docetaxel.

In one embodiment of the process, the amphiphilic block copolymer includes a diblock copolymer having a hydrophilic block (A) and a hydrophobic block (B) linked with each other in the form of A-B structure, and is non-ionic. Additionally, the amphiphilic block copolymer forms core-shell type polymeric micelles in the aqueous environment, wherein the hydrophobic block (B) forms the core and the hydrophilic block (A) forms the shell.

In another embodiment of the process, the hydrophilic block (A) of the amphiphilic block copolymer is a water soluble polymer, and includes at least one selected from the group consisting of polyalkylene glycol, polyvinyl alcohol, polyvinyl pyrrolidone, polyacrylamide and derivatives thereof. Particularly, the hydrophilic block (A) may be at least one selected from the group consisting of polyalkylene glycol, monomethoxypolyalkylene glycol, monoacetoxypolyalkylene glycol, polyethylene-co-propylene glycol, and polyvinyl pyrrolidone. More particularly, the hydrophilic block (A) may be at least one selected from the group consisting of polyethylene glycol, monomethoxypolyethylene glycol, monoacetoxypolyethylene glycol, and polyethylene-co-propylene glycol,

The hydrophilic block (A) may have a number average molecular weight of 500-50,000 daltons, particularly 1,000-20,000 daltons, and more particularly 1,000-10,000 daltons.

The hydrophobic block (B) of the amphiphilic block copolymer is not dissolved in water and may be a biodegradable polymer with high biocompatibility. For example, the hydrophobic block (B) may be at least one selected from the group consisting of polyester, polyanhydride, polyamino acid, polyorthoester, polyphosphazine and derivatives thereof. More particularly, the hydrophobic block (B) may be at least one selected from the group consisting of polylactide, polyglycolide, polycaprolactone, polydioxan-2-one, polylactic-co-glycolide, polylactic-co-dioxane-2-one, polylactic-co-caprolactone and polyglycolic-co-caprolactone. In addition, the above polymers listed as a hydrophobic block (B) may be provided as derivatives thereof substituted with fatty acid groups at the hydroxyl end groups. The fatty acid group may be at least one selected from the group consisting of butyrate, propionate, acetate, stearate, palmitate, tocopherol group, and cholesterol group. Meanwhile, the hydrophobic block (B) may have a number average molecular weight of 500-50,000 daltons, particularly 1,000-20,000 daltons, and more particularly 1,000-10,000 daltons.

In still another embodiment, to form stable polymeric micelles in an aqueous solution, the amphiphilic block copolymer may include the hydrophilic block (A) and the hydrophobic block (B) in a weight ratio of 3:7 to 8:2 (hydrophilic block (A): hydrophobic block (B)), particularly of 4:6 to 7:3. When the proportion of the hydrophilic block (A) is lower than the above range, the polymer may not form polymeric micelles in an aqueous solution. On the other hand, the proportion of the hydrophilic block (A) is higher than the above range, the polymer may be too hydrophilic to maintain its stability.

For example, the organic solvent used in the process is a water miscible organic solvent, and may be at least one selected from the group consisting of alcohol, acetone, tetrahydrofuran, acetic acid, acetonitrile and dioxane. More particularly, the alcohol may be at least one selected from the group consisting of methanol, ethanol, propanol and butanol.

In still another embodiment, although the organic solvent is required to dissolve the polymer and the drug, the organic solvent may be used in the process in a small amount, because the presence of the organic solvent may decrease the micelle stability to accelerate drug precipitation. The organic solvent may be used in an amount of 0.5-30 wt %, particularly 0.5-15 wt %, and more particularly 1-10 wt %, based on the total weight of the composition from which the organic solvent is not removed. When the organic solvent is used in an amount less than 0.5 wt %, it may be difficult to dissolve the drug in the organic solvent. On the other hand, when the organic solvent is used in an amount greater than 30 wt %, drug precipitation may occur during the reconstitution. In a more preferable embodiment, the organic solvent may be used in an amount of 1-6.5 wt %, and particularly 1.5-5 wt % based on the total weight of the composition from which the organic solvent is not removed. Within this range, the time for forming the micelle solution may be shortened.

The poorly water-soluble drug may be dissolved into the organic solvent sequentially or simultaneously with the polymer.

In the method disclosed herein, the drug and the polymer may be simultaneously added to and dissolved into the organic solvent. Otherwise, the polymer may be dissolved first into the organic solvent, followed by the drug, or vice versa. The dissolving a poorly water-soluble drug and an amphiphilic block copolymer into an organic solvent may be carried out at 30° C. or more and less than 80° C., and particularly 50-70° C. Within this range, the time for dissolving the polymer into the organic solvent or the time for forming the micelle solution may be shortened. When the temperature is 80° C. or more, precipitation may occur during formation of the micelle solution.

A particular embodiment of the method for preparing a drug-containing polymeric micelle composition includes:

dissolving an amphiphilic block copolymer into an organic solvent;

dissolving a poorly water-soluble drug into the resultant polymer solution; and

adding an aqueous solution to the resultant mixture of the drug with the polymer to form micelles, wherein the method for preparing a drug-containing polymeric micelle composition requires no separate operation to remove the organic solvent prior to the formation of micelles.

The aqueous solution used in the method may include water, distilled water, distilled water for injection, saline, 5% glucose, buffer, etc.

The polymeric micelle formation may be carried out by adding the aqueous solution at a temperature of 0° C. or more and less than 40° C., and particularly 5-20° C. When the temperature is 40° C. or more, precipitation may occur during formation of the micelle solution.

In still another embodiment, a lyophilization aid may be added to the micelle composition to perform lyophilization, after forming the polymeric micelles. The lyophilization aid may be added in order to allow a lyophilized composition to maintain its cake-like shape. In one embodiment of the lyophilized composition, the lyophilization aid may be at least one selected from the group consisting of sugar and sugar alcohol, and mixtures thereof. The sugar may be at least one selected from lactose, maltose, sucrose, trehalose and a combination thereof. The sugar alcohol may be at least one selected from the mannitol, sorbitol, maltitol, xylitol, lactitol and a combination thereof.

In addition, the lyophilization aid serves to help the polymeric micelle composition to be dissolved homogeneously in a short time during the reconstitution of the lyophilized composition. In this context, the lyophilization aid may be used in an amount of 1-99.8 wt %, and more particularly 10-60 wt %, based on the total weight of the lyophilized composition.

In one embodiment, the polymeric micelle composition may include 0.1-30.0 wt % of a drug in combination with 70-99.9 wt % of an amphiphilic block copolymer having a hydrophilic block and a hydrophobic block, based on the total dry weight of the composition. The above range is adopted considering the encapsulation ratio of a drug and the stability of the polymeric micelles.

The method disclosed herein is simple, reduces the processing time, and is amenable to mass production, because it avoids a need for separate operation to remove the organic solvent. Additionally, the method allows preparation of a drug-containing polymeric micelle composition at low temperature or room temperature, thereby improving the drug stability.

In still another embodiment, the drug-containing polymeric micelle composition may further include pharmaceutical excipients, such as a preservative, stabilizer, hydrating agent or emulsification accelerator, salt for adjusting osmotic pressure and/or buffer, as well as other therapeutically useful materials. The composition may be formulated into various types of oral or parenteral formulations according to a manner generally known to those skilled in the art.

Formulations for parenteral administration may be administered via a rectal, local, transdermal, intravenous, intramuscular, intraperitoneal, subcutaneous route, etc. Typical examples of the parenteral formulations include injection formulations in the form of an isotonic aqueous solution or suspension. In one example embodiment, the composition may be provided in a lyophilized form, which is to be reconstituted with distilled water for injection, 5% glucose, saline, etc., so that it is administered via intravascular injection.

Formulations for oral administration include tablets, pills, hard and soft capsules, liquid, suspension, emulsion, syrup, granules, etc. Such formulations may include a diluent (e.g. lactose, dextrose, sucrose, mannitol, sorbitol, cellulose and glycine), a glidant (e.g. silica, talc, stearic acid and magnesium or calcium salts thereof, as well as polyethylene glycol), etc. in addition to active ingredients. Tablets may include binders, such as magnesium aluminum silicate, starch paste, gelatin, tragacanth, methyl cellulose, sodium carboxymethyl cellulose and polyvinyl pyrrolidine. Optionally, tablets may include pharmaceutically acceptable additives including disintegrating agents such as starch, agar, alginate or sodium salt thereof, absorbing agents, coloring agents, flavoring agents and sweetening agents. Tablets may be obtained by a conventional mixing, granulating or coating process. In addition, typical examples of formulations for parenteral administration include injection formulations, such as isotonic aqueous solutions or suspensions.

The examples and experiments will now be described. The following examples and experiments are for illustrative purposes only and not intended to limit the scope of this disclosure.

The amphiphilic block copolymer used in the method disclosed herein was obtained according to the method as described in International Patent Publication No. WO03/33592.

### Examples 1-3

### Preparation of Polymeric Micelle Compositions Containing Docetaxel

As an amphiphilic block copolymer, monomethoxypolyethylene glycol-polylactide having a number average molecular weight of 2,000-1,766 daltons was prepared. The amphiphilic block copolymer was completely dissolved at 60° C. in the amount as described in Table 1, and 0.08 mL of ethanol was added thereto, followed by thorough mixing. Next, the resultant mixture was cooled to 30° C., docetaxel was added thereto, and the mixture was agitated until a clear solution containing docetaxel completely dissolved therein was obtained. Then, the solution was cooled to 25° C., and 4.0 mL of purified water at room temperature was added thereto, and the reaction mixture was allowed to react until a bluish clear solution was formed, thereby forming polymeric micelles. Then, 100 mg of D-mannitol as a lyophilizing agent was completely dissolved into the solution, and the resultant solution was filtered through a filter with a pore size of 200 nm, followed by lyophilization, to obtain a powdery docetaxel-containing polymeric micelle composition.

TABLE 1

| | Amount (mg) | |
|---|---|---|
| | Docetaxel | Amphiphilic Block Copolymer |
| Example 1 | 20.0 | 380.0 |
| Example 2 | 20.0 | 265.0 |
| Example 3 | 20.0 | 180.0 |

### Examples 4-6

### Preparation of Polymeric Micelle Compositions Containing Paclitaxel

As an amphiphilic block copolymer, monomethoxypolyethylene glycol-polylactide having a number average molecular weight of 2,000-1,766 daltons was prepared. The amphiphilic block copolymer was completely dissolved at 80° C. in the amount as described in Table 2, and ethanol was added thereto, followed by thorough mixing. Next, the resultant mixture was cooled to 30° C., paclitaxel was added to the mixture, and the resultant mixture was agitated until a clear solution containing paclitaxel completely dissolved therein was obtained. Then, the solution was cooled to 25° C., and 5.0 mL of purified water at room temperature was added thereto, and the reaction mixture was allowed to react until a bluish clear solution was formed, thereby forming polymeric micelles. Then, 100 mg of anhydrous lactose as a lyophilizing agent was completely dissolved into the solution, and the resultant solution was filtered through a filter with a pore size of 200 nm, followed by lyophilization, to obtain a powdery paclitaxel-containing polymeric micelle composition.

TABLE 2

| | Amount (mg) | | |
|---|---|---|---|
| | | Amphiphilic Block | |
| | Paclitaxel | Copolymer | Ethanol |
| Example 4 | 30.0 | 570.0 | 95.5 (0.121 mL) |
| Example 5 | 30.0 | 270.0 | 45.0 (0.057 mL) |
| Example 6 | 30.0 | 170.0 | 28.4 (0.036 mL) |

### Comparative Example 1

### Preparation of Docetaxel-Containing Polymeric Micelles Using Solvent Evaporation Process

First, docetaxel and the amphiphilic block copolymer were provided in the same amounts as described in Example 3. Next, 5 mL of ethanol was added to docetaxel and the amphiphilic block copolymer, and the resultant mixture was agitated at 60° C. until the materials were completely dissolved to obtain a clear solution. Then, ethanol was distilled off under reduced pressure at 60° C. for 3 hours using a rotary reduced-pressure distillator equipped with a round bottom flask. The reaction mixture was cooled to 25° C., 4 mL of purified water at room temperature was added thereto and the reaction mixture was allowed to react until a bluish clear solution was obtained, thereby forming polymeric micelles. Then, 100 mg of D-mannitol as a lyophilizing agent was added to the polymeric micelles so that the micelles were completely dissolved, and the resultant mixture was filtered through a filter with a pore size of 200 nm, followed by lyophilization, to obtain a powdery docetaxel-containing polymeric micelle composition.

### Comparative Example 2

### Preparation of Paclitaxel-Containing Polymeric Micelles Using Solvent Evaporation Process

First, paclitaxel and the amphiphilic block copolymer were provided in the same amounts as described in Example 6. Next, 5 mL of ethanol was added to paclitaxel and the amphiphilic block copolymer, and the resultant mixture was agitated at 60° C. until the materials were completely dissolved to obtain a clear solution. Then, ethanol was distilled off under reduced pressure at 60° C. for 3 hours using a rotary reduced-pressure distillator equipped with a round bottom flask. The reaction mixture was cooled to 50° C., 5 mL of purified water at room temperature was added thereto and the reaction mixture was allowed to react until a bluish clear solution was obtained, thereby forming polymeric micelles. Then, 100 mg of anhydrous lactose as a lyophilizing agent was added to the polymeric micelles so that the micelles were completely dissolved, and the resultant mixture was filtered through a filter with a pore size of 200 nm, followed by lyophilization, to obtain a powdery paclitaxel-containing polymeric micelle composition.

### Comparative Example 3

### Preparation of Docetaxel-Containing Polymeric Micelles Using Solvent Evaporation Process

First, docetaxel and the amphiphilic block copolymer were provided in the same amounts as described in Example 3. Next, the amphiphilic block copolymer was completely dissolved at 60° C., and 5 mL of ethanol was added thereto, followed by thorough mixing. The resultant mixture was cooled to 30° C., docetaxel was added thereto and the mixture was further agitated until a clear solution containing docetaxel completely dissolved therein was obtained. Then, ethanol was distilled off under reduced pressure using a rotary reduced-pressure distillator equipped with a round bottom flask. The reaction mixture was cooled to 25° C., 4 mL of purified water at room temperature was added thereto and the reaction mixture was allowed to react until a bluish clear solution was obtained, thereby forming polymeric micelles. Then, 100 mg of D-mannitol as a lyophilizing agent was added to the polymeric micelles so that the micelles were completely dissolved, and the resultant mixture was filtered through a filter with a pore size of 200 nm, followed by lyophilization, to obtain a powdery docetaxel-containing polymeric micelle composition.

### Comparative Example 4

### Preparation of Paclitaxel-Containing Polymeric Micelles Using Solvent Evaporation Process

First, paclitaxel and the amphiphilic block copolymer were provided in the same amounts as described in Example 6. Next, the amphiphilic block copolymer was completely dissolved at 80° C., and 5 mL of ethanol was added thereto, followed by thorough mixing. After that, paclitaxel was added thereto and the mixture was further agitated until a clear solution containing paclitaxel completely dissolved therein was obtained. Then, ethanol was distilled off under reduced pressure using a rotary reduced-pressure distillator equipped with a round bottom flask. The reaction mixture was cooled to 50° C., 5 mL of purified water at room temperature was added thereto and the reaction mixture was allowed to react until a bluish clear solution was obtained, thereby forming polymeric micelles. Then, 100 mg of anhydrous lactose as a lyophilizing agent was added to the polymeric micelles so that the micelles were completely dissolved, and the resultant mixture was filtered through a filter with a pore size of 200 nm, followed by lyophilization, to obtain a powdery paclitaxel-containing polymeric micelle composition.

### Comparative Example 5

### Preparation of Micelles at High Temperature

A docetaxel-containing polymeric micelle composition was prepared in the same manner as described in Example 1, except that the polymeric micelles were formed while maintaining the temperature at 70° C. after adding the ethanol solution. After that, the micelles were lyophilized in the same manner as described in Example 1 to obtain a lyophilized micelle composition.

### Comparative Example 6

### Preparation of Micelles at High Temperature

A paclitaxel-containing polymeric micelle composition was prepared in the same manner as described in Example 6, except that the polymeric micelles were formed while maintaining the temperature at 70° C. after adding the ethanol solution. After that, the micelles were lyophilized in the same manner as described in Example 6 to obtain a lyophilized micelle composition.

### Test Example 1

### Measurement of Amount of Drug Encapsulation

The docetaxel-containing polymeric micelle compositions according to Examples 1-3 and Comparative Examples 1 and 3 were subjected to HPLC as specified in Table 3 to measure the concentration of docetaxel in each composition. Then, the drug content (encapsulation amount) was calculated according to Math Figure. 1. The results were shown in Table 4.

Encapsulation(%)=(measured amount of docetaxel/amount of used docetaxel)×100 [Math Figure. 1]

TABLE 3

| | Condition |
|---|---|
| Mobile Phase | 45% Acetonitrile/55% Water |
| Column | C18, 300A Inner Diameter 4.6 mm, Length 25 cm |
| | (Phenomenex, USA) |
| Detection Wavelength | 227 nm |
| Flow Rate | 1.5 mL/min. |
| Temperature | Room Temperature |
| Infection Volume | 10 μL |

TABLE 4

| | Docetaxel Content (%) |
|---|---|
| Example 1 | 98.7 |
| Example 2 | 101.0 |
| Example 3 | 99.6 |
| Comparative Example 1 | 57.9 |
| Comparative Example 3 | 99.1 |

As can be seen from the above results, the compositions obtained after lyophilization without removing the organic solvent according to Examples 1-3 show a docetaxel content of about 100%. On the other hand, the lyophilized composition obtained after removing the organic solvent at 60° C. according to Comparative Example 1 shows a docetaxel content of about 60%. This demonstrates that docetaxel is decomposed in the polymeric micelles obtained via a solvent evaporation process according to Comparative Example 1 during the evaporation of the organic solvent at high temperature.

In addition, the lyophilized composition obtained after removing the organic solvent at 30° C. according to Comparative Example 3 shows a similar docetaxel content. Therefore, it can be seen that the method disclosed herein provides a similar drug encapsulation amount as compared to the conventional solvent evaporation process, while simplifying the overall process by avoiding a need for separate operation of removing the organic solvent.

### Test Example 2

### Measurement of Particle Size

The paclitaxel-containing polymeric micelle compositions according to Examples 4-6 and Comparative Examples 2 and 4 were reconstituted with 5 mL of saline, and the particle size in each reconstituted composition was measured in aqueous solution using a particle size analyzer (DLS). The results were shown in Table 5.

TABLE 5

| | Particle Size (nm) |
|---|---|
| Example 4 | 29.9 |
| Example 5 | 30.5 |
| Example 6 | 34.3 |
| Comparative Example 2 | 32.3 |
| Comparative Example 4 | 32.4 |

As can be seen from the above results, there is no significant difference in the particle size in aqueous solution between the lyophilized compositions obtained without removing the organic solvent according to Examples 4-6 and the lyophilized compositions obtained after removing the organic solvent according to Comparative Examples 2 and 4.

### Test Example 3

### Stability Test

The paclitaxel-containing polymeric micelle composition according to Example 6 was compared with the paclitaxel-containing polymeric micelle composition according to Comparative Example 2 in terms of the stability in the aqueous solution at 37° C.

Each of the compositions according to Example 6 and Comparative Example 2 was diluted with saline to a paclitaxel concentration of 1 mg/mL. While each diluted solution was allowed to stand at 37° C., concentration of paclitaxel contained in each micelle structure was measured over time by way of HPLC. HPLC was carried out under the same conditions as described in Table 3. The results were shown in Table 6.

TABLE 6

| | Paclitaxel Concentration (mg/mL) | |
|---|---|---|
| Time (hr) | Example 6 | Comparative Example 2 |
| 0 | 1.00 | 1.00 |
| 2 | 1.01 | 0.99 |
| 4 | 0.99 | 0.99 |
| 8 | 0.98 | 0.99 |
| 12 | 1.00 | 0.98 |
| 24 | 0.99 | 0.99 |

As can be seen from the above results, there is no significant difference in the stability in aqueous solution over 24 hours between the lyophilized composition obtained without removing the organic solvent according to Example 6 and the lyophilized composition obtained after removing the organic solvent according to Comparative Example 2.

### Test Example 4

The docetaxel-containing polymeric micelle compositions according to Example 1 and Comparative Example 5 were compared with each other in terms of the docetaxel content and related compound content. The docetaxel content and the related compound content were measured under the same HPLC conditions as described in Table 3 and Table 7, respectively. The results were shown in Table 8.

TABLE 7

| | Condition | |
|---|---|---|
| Mobile Phase | Time (min.) | Water:Acetonitrile |
| | 0-15 | 65:35 → 35:65 |
| | 15-25 | 35:65 → 25:75 |
| | 25-30 | 25:75 → 5:95 |
| | 30-35 | 5:95 → 0:100 |
| | 35-39 | 0:100 |
| | 39-40 | 0:100 → 65:35 |
| | 40-45 | 65:35 |

| | |
|---|---|
| Column | C18, 300A Inner Diameter 4.6 mm, |
| | Length 25 cm |
| | (Phenomenex, USA) |
| Detection Wavelength | 230 nm |
| Flow Rate | 1.0 mL/min. |
| Temperature | Room Temperature |
| Injection Volume | 10 μL |

TABLE 8

| | Content (%) | |
|---|---|---|
| | Docetaxel | Total related compounds |
| Example 1 | 98.7 | 0.97 |
| Comp. Ex. 3 | 88.9 | 5.44 |

As can be seen from the above results, high-temperature preparation causes an increase in the amount of docetaxel-related compounds to five times of the amount of those compounds in the case of low-temperature preparation, resulting in a drop in the docetaxel content. This means that high-temperature processing conditions cause decomposition of a drug.

### Test Example 5

The paclitaxel-containing polymeric micelle composition according to Example 6 was compared with the paclitaxel-containing polymeric micelle composition according to Comparative Example 6 in terms of the paclitaxel content. The paclitaxel content was measured under the same HPLC conditions as described in Table 3. Then, the drug content (encapsulation amount) was calculated according to Math Figure. 2. The results were shown in Table 9.

Encapsulation(%)=[measured amount of paclitaxel/amount of used paclitaxel]×100 [Math Figure. 2]

TABLE 9

| | Paclitaxel content (%) |
|---|---|
| Example 6 | 99.6 |
| Comp. Ex. 6 | 59.9 |

The polymeric micelle composition obtained by adding water to form micelles in the presence of the organic solvent while maintaining a high temperature of 70° C. according to Comparative Example 6 causes precipitation of the drug, paclitaxel. Particularly, the paclitaxel content in Comparative Example 6 is decreased as compared to the paclitaxel content in Example 6 by 40% or more.

### Example 7

As an amphiphilic block copolymer, monomethoxypolyethylene glycol-polylactide having a number average molecular weight of 3,700 daltons was prepared. 2,500 mg of the amphiphilic block copolymer was completely dissolved, and 1,578 mg of ethanol was added thereto, followed by thorough mixing. Next, to the resultant mixture, 500 mg of paclitaxel (PTX) was added at 70° C. (Mixing temperature), and the mixture was agitated at 70° C. (Mixing temperature) until a clear solution containing paclitaxel completely dissolved therein was obtained. Then, the solution was cooled to 20° C. (Micellization temperature), and 45 mL of distilled water for injection at room temperature was added thereto, and the reaction mixture was agitated to react until a clear solution was formed, thereby forming polymeric micelles. Then, 1,250 mg of lactose as a lyophilizing aid was completely dissolved into the solution at 20° C. (Micellization temperature), and the resultant solution was filtered through a filter with a pore size of 200 nm and divided into vials, followed by lyophilization, to obtain a powdery paclitaxel-containing polymeric micelle composition.

### Example 8-19

Paclitaxel-containing polymeric micelle compositions of Examples 8-19 were prepared in the same manner as described in Example 1, except that the amount of ethanol, water and lyophilizing aids, the temperatures, and the kind of lyophilizing aids were applied as described in Table 10.

TABLE 10

| | | | | | Mixing | Micellization | PTX | | Total | | |
|---|---|---|---|---|---|---|---|---|---|---|---|
| | mPEG-PLA | Paclitaxel | EtOH | | Temp. | Temp. | Conc. | Water | volume | lyophilizing aid | |
| | (mg) | (mg) | (mg) | % (w/w) | (° C.) | (° C.) | (mg/mL) | (mL) | (mL) | | (mg) |
| Example 7 | 2,500 | 500 | 1,578 | 3.1% | 70 | 20 | 10 | 45 | 50 | Lactose | 1250 |
| Example 8 | 2,500 | 500 | 1,972.5 | 3.9% | 70 | 20 | 10 | 44 | 50 | Lactose | 1250 |
| Example 9 | 2,500 | 500 | 1,578 | 3.1% | 40 | 20 | 10 | 45 | 50 | Lactose | 1250 |
| Example 10 | 2,500 | 500 | 1,578 | 3.1% | 50 | 20 | 10 | 45 | 50 | Lactose | 1250 |
| Example 11 | 2,500 | 500 | 1,578 | 3.1% | 60 | 20 | 10 | 45 | 50 | Lactose | 1250 |
| Example 12 | 2,500 | 500 | 1,578 | 3.1% | 60 | 5~10 | 10 | 45 | 50 | Lactose | 1250 |
| Example 13 | 2,500 | 500 | 1,578 | 1.9% | 60 | 5~10 | 6 | 78.3 | 83.3 | Lactose | 1250 |
| Example 14 | 2,500 | 500 | 1,578 | 4.6% | 60 | 5~10 | 15 | 28.3 | 33.3 | Lactose | 1250 |
| Example 15 | 2,500 | 500 | 1,578 | 6.1% | 60 | 5~10 | 20 | 20 | 25 | Lactose | 1250 |
| Example 16 | 2,500 | 500 | 1,578 | 1.9% | 60 | 5~10 | 6 | 78.3 | 83.3 | Mannitol | 1250 |
| Example 17 | 2,500 | 500 | 1,578 | 1.9% | 60 | 5~10 | 6 | 78.3 | 83.3 | Threhalose | 1250 |
| Example 18 | 2,500 | 500 | 1,578 | 1.9% | 60 | 5~10 | 6 | 78.3 | 83.3 | Dextrose | 1250 |
| Example 19 | 2,500 | 500 | 1,578 | 1.9% | 60 | 5~10 | 6 | 78.3 | 83.3 | Sucrose | 1250 |

### Comparative Examples 7-9

Paclitaxel-containing polymeric micelle compositions of Comparative Examples 7-9 were prepared in the same manner as described in Example 1, except that the amount of ethanol, water and lyophilizing aid, the temperatures, and the kind of lyophilizing aid were used as described in Table 11.

TABLE 11

| | | | | | Mixing | Micellization | PTX | | Total | | |
|---|---|---|---|---|---|---|---|---|---|---|---|
| | mPEG-PLA | Paclitaxel | EtOH (mg) | | Temp. | Temp. | Conc. | Water | volume | lyophilizing aid | |
| | (mg) | (mg) | (mg) | % (w/w) | (° C.) | (° C.) | (mg/mL) | (mL) | (mL) | | (mg) |
| Comparative Example 7 | 2,500 | 500 | 1,578 | 3.1% | 80 | 20 | 10 | 45 | 50 | Lactose | 1250 |
| Comparative Example 8 | 2,500 | 500 | 1,578 | 3.1% | 60 | 40 | 10 | 45 | 50 | Lactose | 1250 |
| Comparative Example 9 | 2,500 | 500 | 1,578 | 3.1% | 60 | 60 | 10 | 45 | 50 | Lactose | 1250 |

### Test Example 6

The paclitaxel-containing polymeric micelle composition according to Examples 7-19 was compared with the paclitaxel-containing polymeric micelle composition according to Comparative Examples 7-9 in terms of the appearance of micellar solution. Further, the time for agitating the mixture of monomethoxypolyethylene glycol-polylactide, paclitaxel and EtOH at Mixing temperature until a clear solution containing paclitaxel completely dissolved therein was obtained (mixing time), and the time for agitating the reaction mixture until a clear polymeric micelle solution was formed (micellization time) were measured. The results were shown in Table 12.

TABLE 12

| | Appearance of | Mixing Time | Micellization Time |
|---|---|---|---|
| | micellar solution | (hr) | (hr) |
| Example 7 | Clear | 0.5 | 0.5 |
| Example 8 | Clear | 0.5 | 0.5 |
| Example 9 | Clear | 2.0 | 0.5 |
| Example 10 | Clear | 0.5 | 0.5 |
| Example 11 | Clear | 0.5 | 1.0 |
| Example 12 | Clear | 0.5 | 1.5 |
| Example 13 | Clear | 0.5 | 0.5 |
| Example 14 | Clear | 0.5 | 1.5 |
| Example 15 | Clear | 0.5 | 2.0 |
| Example 16 | Clear | 0.5 | 0.5 |
| Example 17 | Clear | 0.5 | 0.5 |
| Example 18 | Clear | 0.5 | 0.5 |
| Example 19 | Clear | 0.5 | 0.5 |
| Comparative | precipitated | 0.5 | — |
| Example 7 | | | |
| Comparative | precipitated | 0.5 | — |
| Example 8 | | | |
| Comparative | precipitated | 0.5 | — |
| Example 9 | | | |

The polymeric micelle compositions obtained by Examples 7-19 showed clear appearance, and short mixing time and micellization time. In contrast, the micelle compositions obtained by Comparative Examples 7-9 were precipitated and failed to form micelle, so the micellization time cannot be measured.

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims. In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1-14. (canceled)

15. A method for preparing a drug-containing polymeric micelle composition in a lyophilized powder form, comprising:
dissolving a poorly water-soluble drug and an amphiphilic block copolymer into an organic solvent;
adding an aqueous solution to the resultant mixture in the organic solvent to form polymeric micelles; and,
dissolving a lyophilization aid in the micelle composition after the formation of the micelles, filtering the obtained composition and carrying out lyophilization of the filtered composition,
wherein the method requires no separate operation to remove the organic solvent,
wherein the organic solvent is used in an amount of 1 to 6.5 wt % based on the total weight of the composition,
wherein the dissolving a poorly water-soluble drug and an amphiphilic block copolymer into an organic solvent is carried out at 30° C. or more and less than 80° C., and
wherein the adding an aqueous solution to the resultant mixture in the organic solvent to form micelles is carried out at 0° C. or more and less than 40° C.

16. The method for preparing a drug-containing polymeric micelle composition according to claim 15, wherein the dissolving a poorly water-soluble drug and an amphiphilic block copolymer into an organic solvent comprises:
dissolving the amphiphilic block copolymer into the organic solvent; and
dissolving the poorly water-soluble drug into the resultant polymer solution.

17. The method for preparing a drug-containing polymeric micelle composition according to claim 15, wherein the drug has a solubility of 100 mg/mL or less to water.

18. The method for preparing a drug-containing polymeric micelle composition according to claim 15, wherein the drug is a taxane anti-cancer agent.

19. The method for preparing a drug-containing polymeric micelle composition according to claim 18, wherein the taxane anti-cancer agent is at least one selected from the group consisting of paclitaxel, docetaxel, 7-epipaclitaxel, t-acetyl paclitaxel, 10-desacetyl-paclitaxel, 10-desacetyl-7-epipaclitaxel, 7-xylosylpaclitaxel, 10-desacetyl-7-glutarylpaclitaxel, 7-N,N-dimethylglycylpaclitaxel, 7-L-alanylpaclitaxel and a mixture thereof.

20. The method for preparing a drug-containing polymeric micelle composition according to claim 15, wherein the amphiphilic block copolymer is a diblock copolymer having a hydrophilic block (A) and a hydrophobic block (B),
the hydrophilic block (A) is at least one selected from the group consisting of polyalkylene glycol, polyvinyl alcohol, polyvinylpyrrolidone, polyacrylamide and derivatives thereof,
the hydrophobic block (B) is at least one selected from the group consisting of polyester, polyanhydride, polyaminoacid, polyorthoester, polyphosphazine and derivatives thereof.

21. The method for preparing a drug-containing polymeric micelle composition according to claim 20, wherein the hydrophilic block (A) has a number average molecular weight of 500-50,000 daltons, and the hydrophobic block (B) has a number average molecular weight of 500-50,000 daltons.

22. The method for preparing a drug-containing polymeric micelle composition according to claim 20, wherein the amphiphilic block copolymer comprises the hydrophilic block (A) and the hydrophobic block (B) in a weight ratio (A:B) of 3:7 to 8:2.

23. The method for preparing a drug-containing polymeric micelle composition according to claim 15, wherein the organic solvent is at least one selected from the group consisting of alcohol, acetone, tetrahydrofuran, acetic acid, acetonitrile and dioxane.

24. The method for preparing a drug-containing polymeric micelle composition according to claim 23, wherein the alcohol is at least one selected from the group consisting of methanol, ethanol, propanol and butanol.

25. The method for preparing a drug-containing polymeric micelle composition according to claim 15, wherein the drug-containing polymeric micelles comprise 0.1-30.0 wt % of the drug and 70-99.9 wt % of the amphiphilic block copolymer having a hydrophilic block and a hydrophobic block, based on the total dry weight of the composition.

26. The method for preparing a drug-containing polymeric micelle according to claim 15, wherein the organic solvent is used in an amount of 1.5 to 5 wt % based on the total weight of the composition,

27. The method for preparing a drug-containing polymeric micelle according to claim 15, wherein the dissolving a poorly water-soluble drug and an amphiphilic block copolymer into an organic solvent is carried out at 50° C. to 70° C.

28. The method for preparing a drug-containing polymeric micelle according to claim 15, wherein the adding an aqueous solution to the resultant mixture in the organic solvent to form micelles is carried out at 5° C. to 20° C.
